(19) United States
(12) Patent Application Publication
(10) Pub. No.: US 2020/0101189 A1
(43) Pub. Date: Apr. 2, 2020

(54) DEVICE AND METHOD FOR CONTROLLING SCENT

(71) Applicant: Wildgame Innovations, LLC, Grand Prairie, TX (US)

(72) Inventors: Brandon Roach, Plano, IL (US); Ou Lei, Plano, IL (US); Teck Khoon Ng, Plano, IL (US); Tin Hung Ngai, Plano, IL (US); Siu Wa Ricky Wu, Plano, IL (US); Clark McCune, Plano, IL (US)

(21) Appl. No.: 16/589,847

(22) Filed: Oct. 1, 2019

Related U.S. Application Data
(60) Provisional application No. 62/739,957, filed on Oct. 2, 2018.

(51) Int. Cl. A61L 9/22; A01M 31/00; F16M 13/02

(52) U.S. Cl. CPC A61L 9/22; A01M 31/00; A61L 2209/15; A61L 2202/14; A61L 2202/16; F16M 13/02

(57) ABSTRACT

A system, a method, and a device that control or eliminate the scent of an object in an application domain. The device has a plasma ion generator that includes an air intake that receives air molecules, a plasma ion generator head that disassociates molecular bonds in the received air molecules to create plasma ions, and a plasma ion ejector that directs the plasma ions from the plasma ion generator head in a predetermined direction, wherein the plasma ion generator applies the plasma ions to airborne particles in the application domain to eliminate or control the scent of the object.

### CROSS-REFERENCE TO PRIOR APPLICATION

This application claims priority to and the benefit thereof from U.S. Provisional Patent Application No. 62/739,957, filed Oct. 2, 2018, titled “Device and Method for Controlling Scent,” the entirety of which is hereby incorporated herein by reference.

### FIELD OF THE DISCLOSURE

The disclosure relates generally to a system, method and device for controlling scent, and more particularly, a system, method and device for generating and applying plasma ions for controlling scent, including eliminating scent in open or closed areas.

### BACKGROUND OF THE DISCLOSURE

In an effort to reduce animal scent, various technologies have been employed over the years, including, among other things, masking scents with naturally occurring or artificially created substances, anti-microbial agents or ozone de-scenting. These technologies, however, tend to be ineffective as they only mask undesirable scents and, in the case of ozone de-scenting, precautions might be necessary since ozone can be harmful if ingested, even in low quantities, and because it has a high oxidative effect on clothing, equipment and structures. There exists an unmet need for a de-scenting solution that can be easily and effectively used in the field or in closed spaces, without the drawbacks commonly encountered with known scent control methodologies.

### SUMMARY OF THE DISCLOSURE

A method, a system, and a device are provided for controlling scent, and more particularly, a method, a system, and a device are provided for generating and applying plasma ions for controlling scent, including eliminating scent. The method, system and device can provide electronic scent elimination with positive ion particle flow, negative ion particle flow, or positive and negative ion particle flow that is similarly found in nature, including forests or mountains.

According to a non-limiting aspect of the disclosure, a de-scenting system is provided for controlling or eliminating a scent of an object in an application domain. The de-scenting system embodiment comprises a plasma ion generator that includes an air intake that receives air molecules, a plasma ion generator head that disassociates molecular bonds in the received air molecules to create plasma ions, a plasma ion ejector that directs the plasma ions from the plasma ion generator head in a predetermined direction, and a fan that moves the surrounding air molecules to the air intake, and that moves the plasma ions through the plasma ion ejector in the predetermined direction, wherein the plasma ions are applied to airborne particles in the application domain to eliminate or control the scent of the object. The object can be a person, clothing or equipment employed in a hunting sport.

The plasma ion generator can comprise a controller, a plasma ion driver, and a motion detector. The controller can instruct the plasma ion driver to supply power to the plasma ion generator head based on a motion detection signal received from the motion detector.

The plasma ion generator can comprise a plasma ion generator circuit that includes a controller that generates a pulse width modulation (PWM) signal and a fan control signal, a plasma ion driver that supplies power to the plasma ion generator head based on the pulse width modulation (PWM) signal, and a motor driver that supplies power to the fan based on the fan control signal.

The plasma ions can be applied to a surface of the object in the application domain, and the plasma ions can deodorize the object to camouflage the scent of the object.

The predetermined direction can be downward when the plasma ion generator is positioned above the object, or upward when the plasma ion generator is located on a structural surface.

The plasma ion generator can comprise an attachment member that attaches to a tree.

The plasma ion generator head can include a canode pairing=that is, a cathode and anode pairing. The canode pairing can comprise a needle type or brush type anode or cathode. The canode pairing can comprise carbon fiber.

According to a further non-limiting aspect of the disclosure, a de-scenting system is provided for controlling or eliminating a scent in an application domain, where the de-scenting system comprises a plasma ion generator that includes a motion detector that detects movement of an object in or near the application domain and generates a motion detection signal, an air intake that receives air molecules, a plasma ion generator head that disassociates molecular bonds in the received air molecules to create plasma ions, a plasma ion ejector that directs the plasma ions from the plasma ion generator head in a predetermined direction, a plasma ion driver that supplies power to the plasma ion generator head, a controller that generates a pulse width modulation (PWM) signal to drive the plasma ion driver based on the motion detection signal, and a fan that moves the surrounding air molecules to the air intake, and that moves the plasma ions through the plasma ion ejector in the predetermined direction, wherein the plasma ions are applied to airborne particles in the application domain to eliminate or control the scent. The predetermined direction can be upward when the plasma ion generator is located on a structural surface. The de plasma ion generator head can include a canode pairing, wherein the canode pairing can comprise a needle type or brush type anode or cathode, or carbon fiber.

According to a still further non-limiting aspect of the disclosure, a method is provided for controlling or eliminating scent an object in an application domain. The method comprises receiving air molecules through an air intake, disassociating molecular bonds in the received air molecules to create plasma ions, directing the plasma ions in a predetermined direction, moving the plasma ions through a plasma ion ejector in the predetermined direction, and applying the plasma ions to airborne particles in the application domain to eliminate or control the scent of the object. The method can further comprise applying the plasma ions to airborne particles in the application domain to de-scent the object to camouflage the scent of the object from wild game.

According to a still further non-limiting aspect of the disclosure, a de-scenting system is provided that eliminates odors or reduces or diminishes a scent of an object from animals. The de-scenting system comprises a plasma ion generator device that applies the plasma ions to a surface of the object in an application domain, wherein the plasma ions deodorize the object to camouflage the scent of the object. The plasma ion generator device can include an attachment member that can be attached to an article such as a tree, and that can position the plasma ion generator device to apply the plasma ion to the object. The object can be a person, a clothing or an equipment employed in a hunting sport. The predetermined direction can be downward when the plasma ion generator device is positioned above the object. The plasma ion generator can include a power supply. The power supply can include a battery pack comprising lithium-ion cells. The plasma ion generator can include a high voltage coil transformer that converts a low voltage signal received from the power supply into a high voltage signal. The high voltage alternating current can be supplied to the plasma ion generator head to disassociate molecular bonds in air molecules. The plasma ion generator device can include a power management circuit. The plasma ion generator can comprise a computing device. The computing device can generate a pulse width modulated signal that drives the high voltage coil transformer. The plasma ion generator can comprise a user interface or display. The user interface can include one or more actuators. The display can include a light emitting diode. The one or more actuators can be connected to the computing device and control a mode of operation of the plasma ion generator head.

Additional features, advantages, and embodiments of the disclosure may be set forth or apparent from consideration of the following detailed description and drawings. Moreover, it is noted that both the foregoing summary of the disclosure and the following detailed description are exemplary and intended to provide further explanation without limiting the scope of the disclosure.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced.

FIG. 1 shows a non-limiting implementation of a de-scenting system constructed according to the principles of the disclosure.

FIG. 2 shows another non-limiting implementation of the de-scenting system, constructed according to the principles of the disclosure.

FIG. 3 shows an example of a cathode-anode pairing the can be included in the de-scenting system in FIG. 1 or 2.

FIGS. 4A and 4B show an example of a de-scenting process in which undesirable or harmful particles are eliminated by the de-scenting system in FIG. 1 or 2.

FIGS. 5A and 5B show respective side-front and side-back perspective views of an embodiment of a plasma ion (PI) generator constructed according to the principles of the disclosure.

FIG. 6 shows a side-top perspective view of another embodiment of the PI generator constructed according to the principles of the disclosure.

FIG. 7 shows the PI generator of FIG. 6 in an open configuration.

FIG. 8 shows a view of a bottom portion of the PI generator of FIG. 6, with one or more components removed.

FIG. 9 shows an embodiment of a PI generator circuit, constructed according to the principles of the disclosure.

FIG. 10 shows an embodiment of a PI driver circuit that can be included in the PI generator circuit of FIG. 9.

FIG. 11 shows an example of the difference in the amount of ion output between a brushes type and a needle type canode.

FIG. 12 shows an example of a de-scenting process that can be carried out by the PI generator circuit of FIG. 9.

The present disclosure is further described in the detailed description and drawings that follows.

### DETAILED DESCRIPTION OF THE DISCLOSURE

The disclosure and its various features and advantageous details are explained more fully with reference to the non-limiting embodiments and examples that are described or illustrated in the accompanying drawings and detailed in the following description. It should be noted that features illustrated in the drawings are not necessarily drawn to scale, and features of one embodiment can be employed with other embodiments as those skilled in the art would recognize, even if not explicitly stated. Descriptions of well-known components and processing techniques can be omitted so as to not unnecessarily obscure the embodiments of the disclosure. The examples used are intended merely to facilitate an understanding of ways in which the disclosure can be practiced and to further enable those skilled in the art to practice the embodiments of the disclosure. Accordingly, the examples and embodiments should not be construed as limiting the scope of the disclosure. Moreover, it is noted that like reference numerals represent similar parts throughout the several views of the drawings.

A de-scenting solution is provided for reducing or eliminating animal odor or scent, including human odor or scent, in an open or closed area. The term “scent” as used in this disclosure means odor and/or scent. The olfactory system, or sense of smell, is the part of the sensory system used for smelling. Most mammals have a main olfactory system and an accessory olfactory system. The main olfactory system detects airborne particles, while the accessory system senses fluid-phase substances. The de-scenting solution focuses primarily on airborne particles, but can also be used to de-scent fluid-phase substances. The de-scenting solution can be employed to reduce or eliminate airborne particles in an open area such as when observing or hunting game, or in a closed area such as a room, including, for example, a bathroom, a locker room, a hospital room, a bedroom, an office, or any other space that could benefit from the technological solution.

In one non-limiting application, the de-scenting solution can provide electronic control or elimination of human scent in the field such as when hunting game, or for getting close enough to an animal to observe or photograph it. Animals have an acute sense of smell and are capable of recognizing a human scent or any other scent at long distances. Scent control is an important aspect of hunting animals that rely on their sense of smell for survival. This is especially true for bow hunting or muzzleloader hunting where the hunter must be in close range to the animal. To avoid such recognition, various scent control methodologies are employed by hunters, ranging from playing-the-wind to masking or reducing scent.

Playing-the-wind has many drawbacks that make it either impractical or ineffective. For instance, wind velocity or wind intensity can change frequently and drastically during a hunt, requiring the hunter to frequently change location or to find a location that limits scent travel, but which may be highly undesirable, ineffective, impractical, or just plain dangerous.

In addition, or as an alternative to playing-the-wind, many hunters try to mask their scent and that of their equipment. Their objective is to mask as much of their human scent as possible through scent-killing soaps, cover scents or animal attractants. Hunters commonly employ masking agents like pine needles, cedar foliage, balsams and other evergreens, apple cider, or animal urine. However, because animals such as deer have senses of smell that are hundreds of times that of humans, they can still sense human scent through the masking agent.

Activated carbon-lined hunting garments and coverings, anti-microbial undergarments and ozone de-scenting have also been used to control human scent. These technologies, however, are prone to adsorbing unintended scents that can scare off game, such as, for example, gas scents that might be picked up by the hunter's garments while filling gas, cooking or smoking. In the case of ozone de-scenting, precautions might be necessary since ozone can be harmful if ingested, even in low quantities, and has a high oxidative effect on clothing and equipment.

In another non-limiting application, the de-scenting solution can provide electronic scent control or elimination in closed or semi-closed spaces such as, for example, in bathrooms, basements, animal pens, locker rooms, saunas, bedrooms, offices, or hospital rooms. Such scents can be unpleasant, a common source of embarrassment, or, in some instances, harmful to occupants in the closed area. These scents are typically masked through the use of artificial scents like floral sprays, or reduced through comprehensive cleaning regimens or ventilation. However, because olfactory systems of animals, including humans, can be very sensitive and still smell the underlying scents, such remedial efforts tend to be ineffective, resource-intense, expensive, or, in some instances, can expose occupants or the environment to hazardous substances, such as those commonly found in cleaning agents.

The de-scenting solution can effectively reduce or eliminate animal scent, including human scent, without having to use resource-intense, costly, or hazardous methodologies. The de-scenting solution includes a method, a system, or a device that can be deployed in the field or a closed (or semi-closed) environment to control or eliminate scent by applying plasma ions to airborne particles. As noted earlier, the de-scenting solution can also be employed with fluid-phase substances.

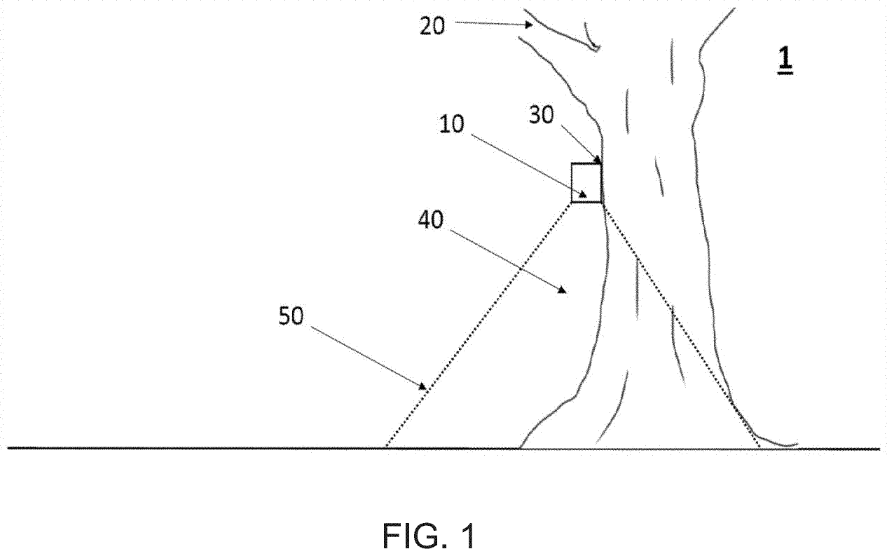

FIG. 1 shows an example of a plasma ion (PI) de-scenting system, according to the principles of the disclosure. The PI de-scenting system includes a PI generator 10 that can be employed in an open area 1. As seen in FIG. 1, the PI generator 10 can be attached to a stationary object 20 such as a tree, or any other object that can support and hold the PI generator 10 in a desired location and position. The PI generator 10 can be attached to the object 20 by an attachment member 30. The attachment member 30 can be attached to the PI generator 10, or integrally formed as part of the PI generator 10, such as a housing of the PI generator 10. The attachment member 30 can include a bracket, a plate, a strap, a rope, a nail, a screw, a rod, a pin, a hook, a loop, or any other mechanism that can secure the PI generator 10 to the object 20. The PI generator 10 can be positioned to emit a PI stream 40 in an application domain 50. The PI stream 40 can include de-scented air. The PI stream can include positively or negatively charged ions, including, for example, positively charged hydrogen H⁺ ions or negatively charged oxygen O₂⁻ ions.

The PI generator 10 can be made adjustable such that the PI stream 40 can be adjusted to match and envelope the area or volume of the application domain 50. The PI stream 40 can be adjusted to have any shape that matches the shape, size, or volume of the application domain 50. Where one PI stream 40 might be insufficient, additional PI generators 10 can be employed. The PI stream 40 can be adjusted to account for wind conditions in the application domain 50. The PI stream 40 can have a conical shape, a rectangular shape, a cylindrical shape, a spherical shape, a semi-spherical shape, or any other shape that can be accommodated in the space 1. One or more additional PI generators 10 can be employed, and each of their PI streams can be adjustable to overlap or supplement the PI stream 40 to adjust shape, size, intensity or direction of the PI stream(s) 40 to envelope the application domain 50. The PI generator 10 can optionally include an ozone O₃ generator (not shown) that can generate and emit ozone.

The PI generator 10 produces and emits the PI stream 40. The PI generator 10 can intake surrounding air and disassociate molecular bonds in the air molecules by subjecting the molecules through one or more electromagnetic fields or heating the molecules to the point where ionized molecules become increasingly electrically conductive. The PI generator 10 can then emit the ionized molecules (“ions”) in the PI stream 40. The PI stream 40 can envelop the application domain 50, treating everything in the application domain 50 with large quantities of ions (e.g., thousands, millions, or more ions). The emitted ionized molecules, which can include ionized hydrogen (H) and ionized oxygen (O₂) molecules, have positive and negative charges. The positively charged hydrogen H⁺ ions are missing an electron, and the negatively charged oxygen O₂⁺ ions have an extra electron, resulting in unstable conditions.

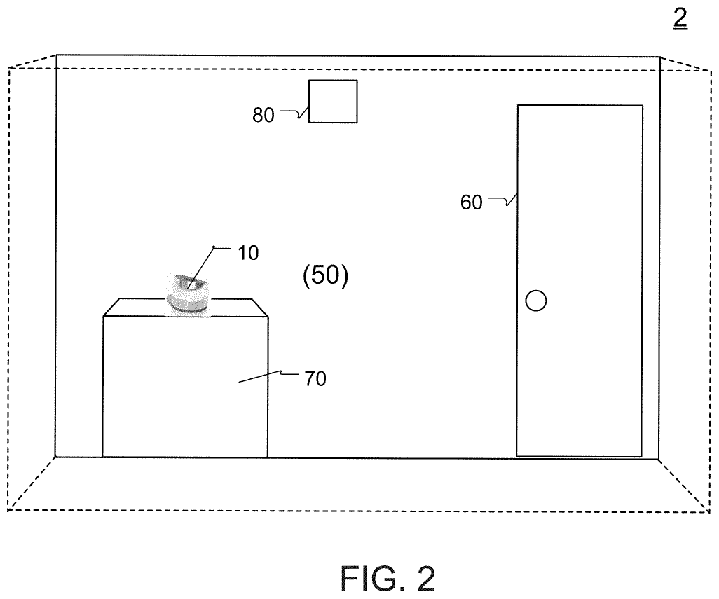

FIG. 2 shows a non-limiting implementation of the de-scenting system in a closed or semi-closed area, according to the principles of the disclosure. The closed or semi-closed area can include a room 2 such as, for example, a bathroom, basement, animal pen, locker room, sauna, bedroom, office, hospital room, or any other space that could benefit from application of the de-scenting system. The room 2 can include a plurality of walls 50, a ceiling, a floor, or a door 60. The room 2 can include a structure 70 such as, for example, a bathroom sink, toilet, or furniture. The room 2 can include one or more PI generators 10 that can be located anywhere in the room 2, and can be positioned in a location(s) for optimal performance, such as, for example, on the structure 70 or on a wall 50.

In addition to the PI generator 10, the de-scenting system can include an optional scent monitor 80. The PI generator 10 and scent monitor 80 can each include a communicating device that can be configured to communicate with each other over a communication link. The scent monitor 80 can measure concentration levels of a particular substance or organism in the room 2. The scent monitor 80 can be configured to detect and measure the particular substance or organism in parts per million (ppm) relative to the air in the room 2. For instance, the scent monitor 80 can be configured to measure hydrogen sulfide, methane, methyl mercaptan, or ammonia. The scent monitor 80 can include an OMX-ADM odor meter. The PI generator 10 can receive a scent level signal from the scent monitor 80 and, based on the scent level signal, can turn ON/OFF or adjust the PI stream emitted by the PI generator 10. The scent level signal can include real-time or historical information about the concentration value of the detected substance or organism, or distribution or dispersion vector of the substance or organism in the room 2.

Optimal performance of the de-scenting system, including the PI generator 10, can depend on factors such as, for example, the location where a target scent is likely to originate, or where occupants are likely to spend most of their time while in the room 2. The PI generator 10 can be positioned to optimize de-scenting in the room 2.

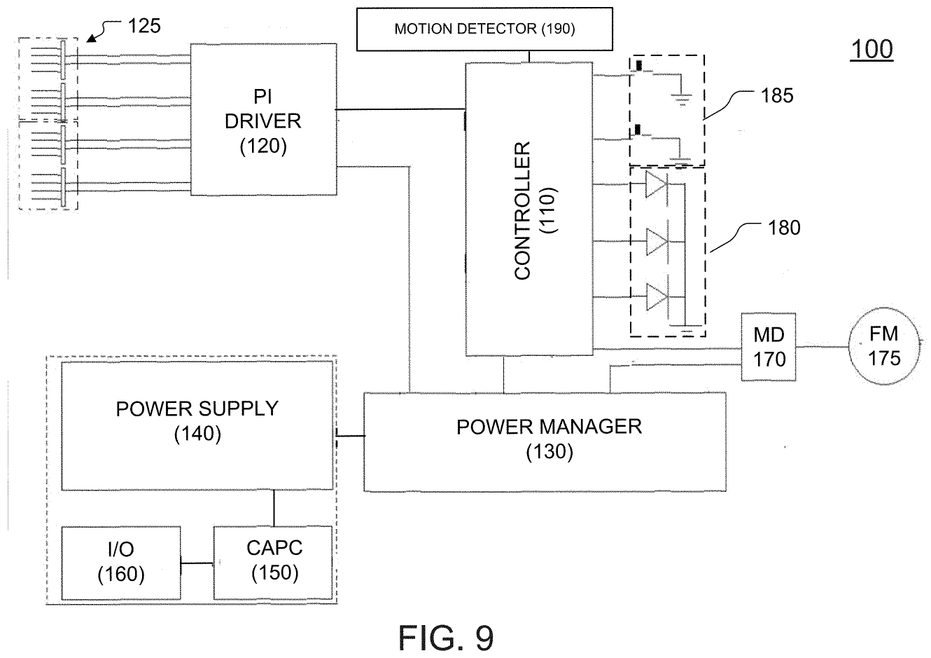

The PI generator 10 can include a motion detector 190 (shown in FIG. 9) that can detect the presence of an occupant in the room 2. Based on motion of an occupant in the room 2 detected by the motion detector 190, the PI generator 10 can be configured to turn ON or OFF, or to adjust characteristics of the PI stream 40 such as, for example, direction, intensity, or spread (e.g., width or height) of the PI stream 40, thereby determining and controlling the application domain 50 (shown in FIG. 1) for optimal de-scenting. When an occupant enters the room 2, such as through the door 60, the motion detector 190 (shown in FIG. 9) can detect the occupant's presence and the PI generator 10 can be turned ON. The PI generator 10 can run during the entire (or less than entire) time the occupant is present in the in the room 2. The PI generator 10 can be configured to turn OFF after a predetermined amount of time passes during which no occupant is detected in the room 2, thereby saving energy. The predetermined amount of time can be set to, for example, 1 minute, 5 minutes, 10 minutes, or any other amount of time sufficient for de-scenting, balanced with energy usage considerations.

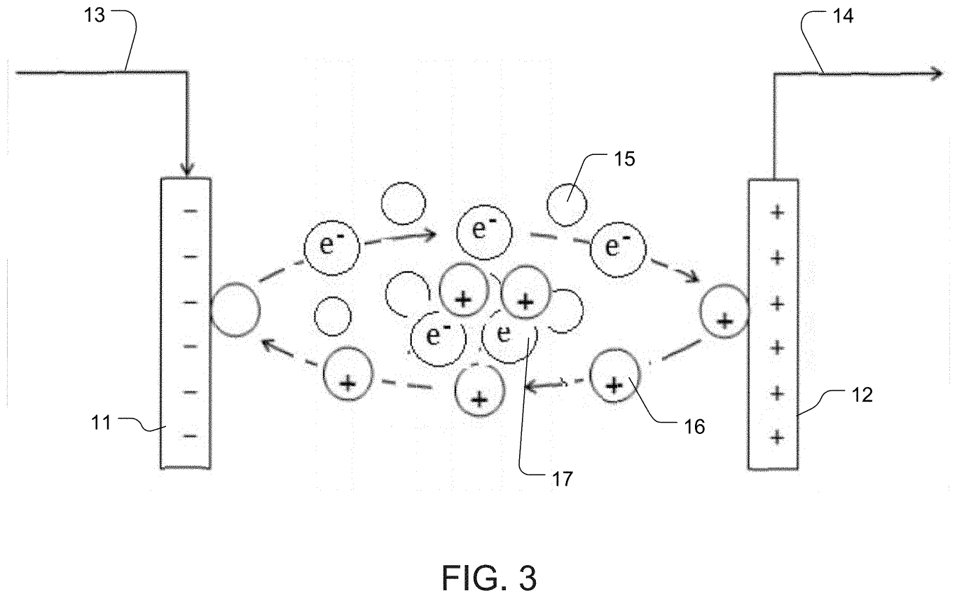

FIG. 3 shows an example of a cathode-anode (canode) pairing 11, 12 that can be included in the PI generator 10 to disassociate molecular bonds in air, such as, for example, water molecules (H₂O) and oxygen molecules (O₂) to create positively or negatively charged ions. The canode 11, 12 can be connected to a power supply (not shown) via lines 13, 14, respectively, to create an electrical field that disassociates molecular bonds in molecules between the cathode 11 and anode 12. The voltage potential between the cathode 11 and anode 12 can be, for example, about 6500 v. The voltage potential can be higher or lower than 6500 v. The canode 11, 12 can disassociate H₂O molecular bonds into positively charged hydrogen H⁺ ions and negatively charged oxygen O₂⁺ ions.

As seen in FIG. 3, the space between the cathode 11 and anode 12 can include neutral particles 15, charged ions 16, or a charged aggregate 17. A neutral particle 15 carrying an electron can be attracted to the anode 12 and travel in the direction of the anode until it contacts the anode 12, where the electron is removed from the neutral particle 15 to create a positively charged ion 16. The positively charged ion 16 is then attracted to the cathode 11 and travels in the direction of the cathode until it contacts the cathode 11, where an electron is added to the positively charged ion 16 to neutralize and, thereby, create a neutral particle 15. The charged aggregate 17 can include an aggregate of charged ions 16 that can combine to produce a high energy aggregate sufficient to kill organisms such as, for example, bacteria, viruses, or fungi.

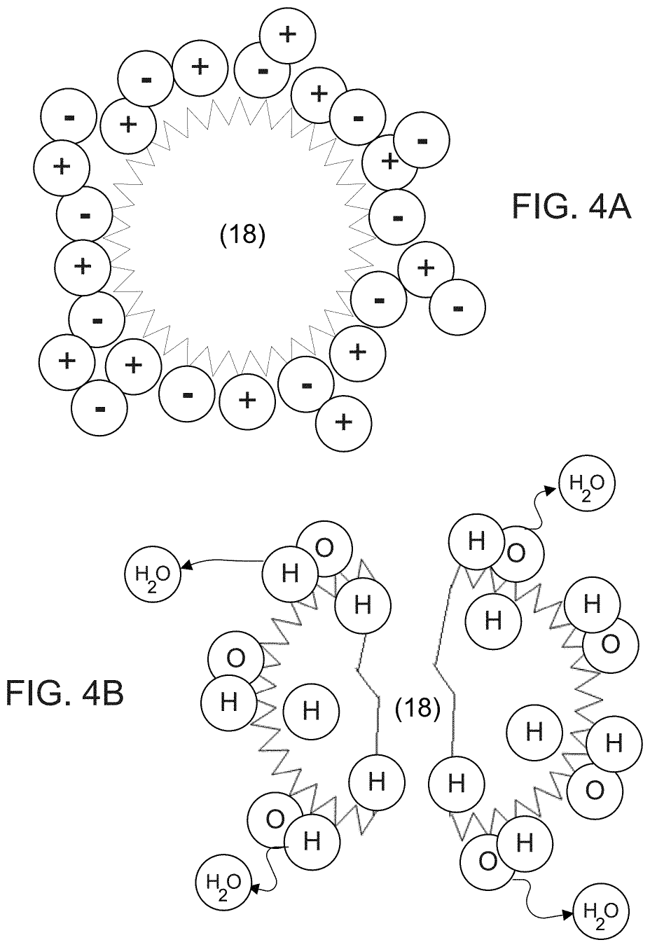

FIGS. 4A and 4B show an example where positively charged hydrogen H+ ions and negatively charged oxygen O₂ ions are generated by the PI generator 10 and emitted to contact surfaces of an airborne scent particle 18 that can include a substance such as a gas, or an organism such as, for example, bacteria, virus, or fungus. As the H⁺ and O₂⁻ ions contact with the scent particle 18, the charged H⁺ and O₂⁻ ions adhere to the surfaces of the particle 18. The charged H⁺ and O₂⁺ ions cluster together as they attach on the surfaces, causing a chemical reaction that results in creation of highly reactive hydroxyl (OH) radicals. The hydroxyl radical will take a hydrogen molecule from the cell wall of the organism, killing it in the process. Resultantly, scents can be quickly and effectively eliminated by the canode 11, 12 (shown in FIG. 3).

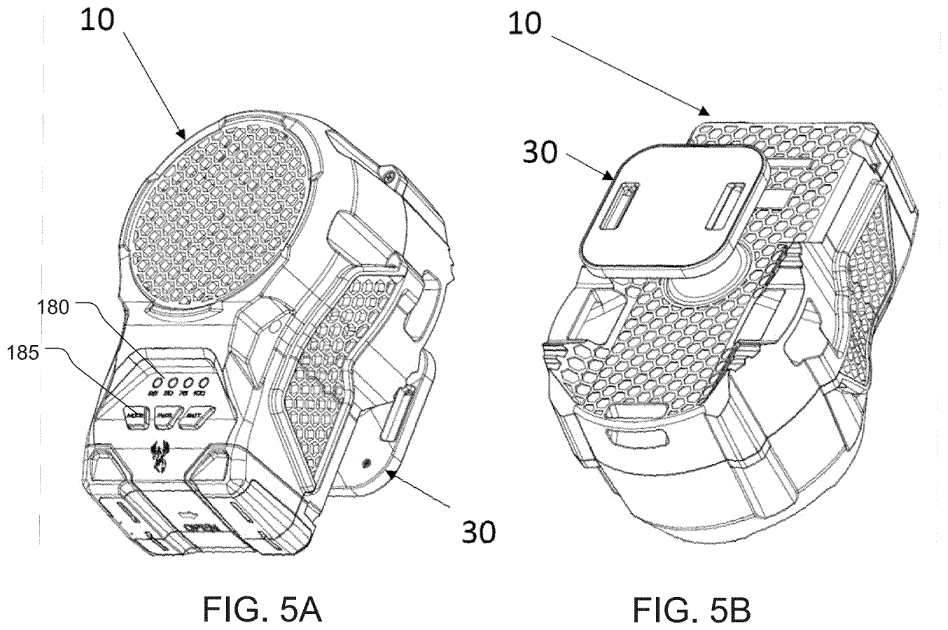

FIGS. 5A and 5B show a side-front perspective view and a side-back perspective view of a non-limiting embodiment of the PI generator 10, according to the principles of the disclosure. The PI generator 10 can include a housing that is designed to have a shape, size, color, or texture to match the object 20 in the open space 1 (shown in FIG. 1). For instance, the housing of the PI generator 10 can be designed to match the shape, color or texture of a portion of a tree such that when the PI generator 10 is attached to the tree, the PI generator 10 can be camouflaged with its surroundings, making it practically invisible to animals. The housing can be attached to or formed integrally with the attachment member 30. The attachment member 30 can include openings that can receive an article such as a strap to attach to the object 20 (shown in FIG. 1).

The PI generator 10 can include a plurality of air intake openings to take in surrounding air and a plurality of PI ejection openings to emit and direct the PI stream 40 (shown in FIG. 1). The PI generator 10 can include a display 180 and a user interface 185. The display 180 and user interface 185 can be combined into a single device such as, for example, a touch-screen display. The display 185 can include a liquid crystal display (LCD), a light emitting diode (LED) display, a quantum dot LED (QLED), or a plurality of light emitting elements such as LEDs (e.g., 4 LEDs). The user interface 185 can include a button, a keypad, a keyboard, a joy stick, a toggle switch, or voice response system (VRS) that can respond to voice commands. The display 180 can display the operating status of the PI generator 10, including an operating mode, status of the PI stream (e.g., PI stream 40 in FIG. 1) or power supply level. The user interface 185 can be actuated to turn ON/OFF the PI generator 10, to control a PI mode or to check the status of the power supply such as a battery. The PI mode can include, for example, a distance mode, a fan speed mode, a PI stream rate mode, or a PI stream spread mode.

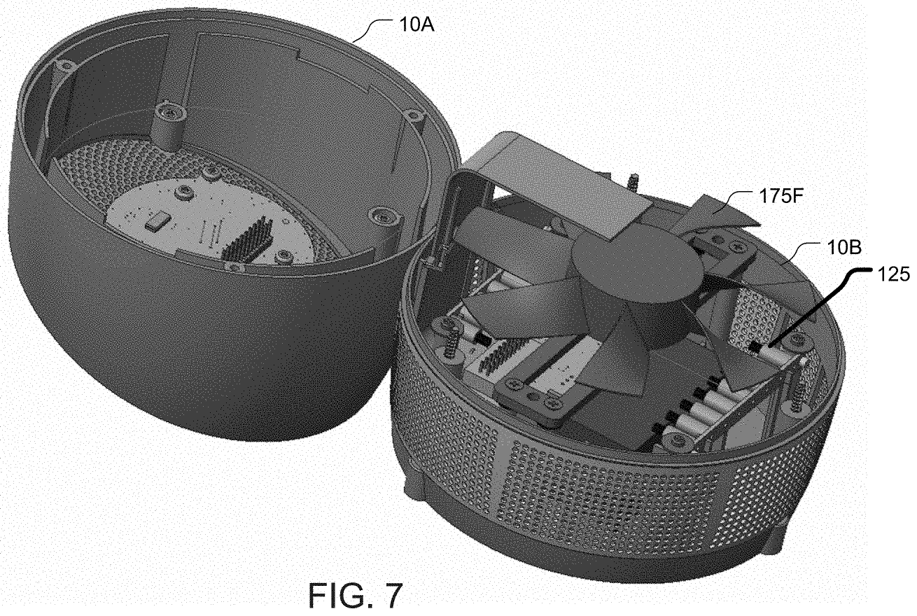

The PI distance mode can include a plurality of distances (e.g., 5 ft, 10 ft, 25 ft) for the operating range of the PI generator 10, which can be selected by the user via the user interface 185 to control how far the PI stream 40 should reach and effectively eliminate scents. The PI stream rate mode can allow the user to select (via the user interface 185) the rate at which the PI generator 10 generates and emits ions in the PI stream 40. The PI stream spread mode can allow the user to select the angular spread of the PI stream 40, such that the PI stream 40 can be set to spread at a desired angular rate with respect to distance (e.g., the PI stream 40 covers a circular radius of 5 ft at a distance of 10 ft) or time to provide coverage for the entire application domain 50 (shown in FIG. 1). The fan mode can allow the user to select the operating speed of the fan 175 (shown in FIG. 7), such as, for example, LOW, MEDIUM, HIGH.

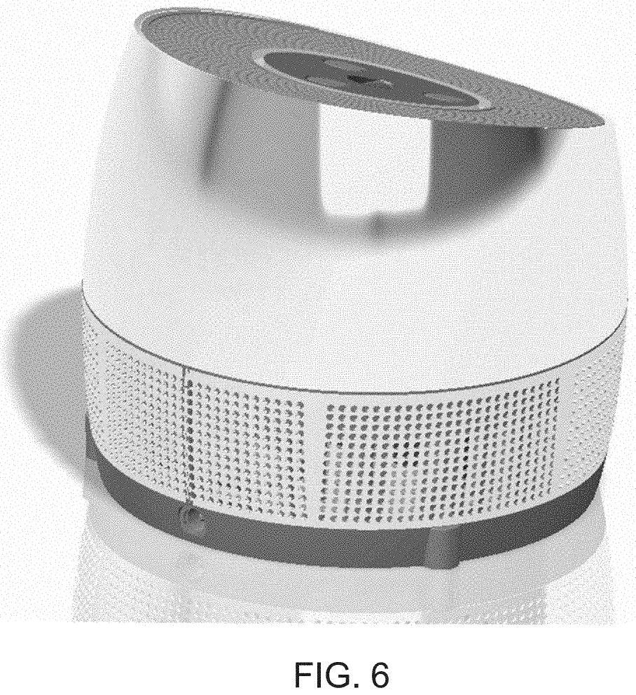

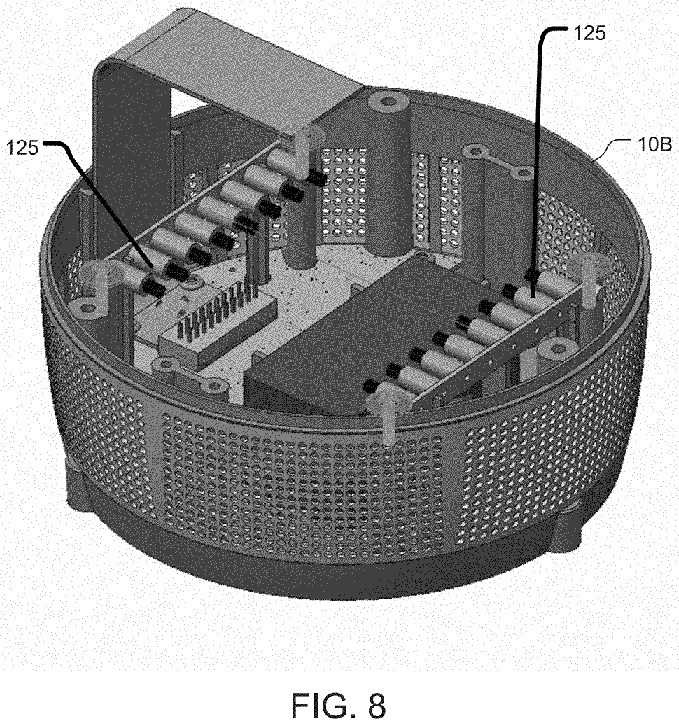

FIGS. 6-8 show various views of another non-limiting embodiment of the PI generator 10, according to the principles of the disclosure. FIG. 6 shows a side view of the PI generator 10 in an operational configuration; FIG. 7 shows a view of the PI generator 10 in an open configuration; and, FIG. 8 shows a view of a bottom portion of the PI generator 10, with components removed to show canode pairings 125. This embodiment of the PI generator 10 can be designed to be aesthetically appealing to users and not necessarily to blend with the environment for camouflage. The canode pairings 125 can include a plurality of cathode-anode pairs. The cathodes or anodes in the canode pairing 125 can include a carbon brush or needle.

The PI generator 10 can include a quasi-cylindrical-shaped housing with a plurality of air intake openings to take in surrounding air and a plurality of PI ejection openings to emit and direct a PI stream, such as the PI stream 40, shown in FIG. 1. The housing can have any shape or size, depending on the application or aesthetic appeal to the user. The PI generator 10 can include a top portion 10A and a bottom portion 10B. The air intake openings can be formed in the bottom portion 10B (or top portion 10A), and the PI ejection openings can be formed in the top portion 10A (or bottom portion 10B). The PI ejection openings can be configured to direct the PI stream upward when the PI generator 10 is placed on a horizontal planar surface. The canode pairings 125 can be affixed in the bottom portion 10B, as shown in FIG. 7, or in the top portion 10A. The PI generator 10 can include a fan 175 (shown in FIG. 7), which can be affixed proximate the canode pairings 125. The fan 175 can be affixed in the bottom portion 10B, as shown in FIG. 7, or in the top portion 10A.

FIG. 9 shows a non-limiting embodiment of a PI generator circuit 100 that can be included in the PI generator 10, according to the principles of the disclosure. The PI generator circuit 100 can be included in the PI generator 10 according to the embodiment shown in FIGS. 1, 5A and 5B, the embodiment shown in FIGS. 2 and 6-8, or any other embodiment constructed according to the principles of the disclosure. The PI generator circuit 100 can include a controller 110, a plasma ion (PI) driver 120, a PI generator head 125, a power manager 130, a power supply 140, a charge and protect circuit (CAPC) 150, an input/output (I/O) interface 160, a motor driver (MD) 170, a fan motor (FM) 175, the display 180, and user interface 185. The PI generator circuit 100 can include a motion detector 190, which can be optional. The PI generator circuit 100 can include a transmitter or a receiver (not shown). The PI generator circuit 100 can be configured to connect to a network, such as, for example, a residential local area network (LAN) via a communication link.

The PI generator circuit 100 can include a storage device (not shown), including, for example, a random-access memory (RAM) or a read-only memory (ROM). The storage device can be included in the controller 110. The storage device contains a computer-readable medium.

The controller 110 can include a communicating device or a computing device. The controller 110 can include a microcontroller unit (MCU).

The PI driver 120 can include a high-voltage coil transformer (not shown) that can convert low voltage power (e.g., 12 Volts) to high-voltage power (e.g., 6500 Volts). The PI driver 120 can receive a driver signal from the controller 110, which can include a pulse-width modulation (PWM) signal, to drive the high-voltage coil transformer and control the PI generator head 125. The PI driver 120 can supply the high-voltage power to the PI generator head 125 over a plurality of power supply lines. The PI generator head 125 can include canode pairings that create an electromagnetic field to ionize molecules. The canode pairings can include one or more cathodes 11 paired with one or more anodes 12 (shown in FIG. 3).

The PWM signal can be generated by the controller 110 and supplied to the PI driver 120 to drive the PI generator head 125. The frequency of the PWM signal applied to the PI generator head 125 can range between, for example, about 10 KHz and about 20 KHz. The frequency can be less than 10 KHz or more than 20 KHz.

The voltage of the electrical signal supplied to the PI generator head 125 can be, for example, about +/−6500 v. The voltage can by higher than +/−6500 v or lower than +/−6500, such that the amount of negative ion and positive ions can be greatly varied in the range of 6 million pcs/cm to 14 million pcs/cm respectively.

The PI generator head 125 can be configured with a current draw in the range of, for example, about 700 mAh to about 800 mAh at a 12 Volt input at the voltage transformer. The current draw can be less than 700 mAh or greater than 800 mhAh.

The PI generator head 125, when driven by the PWM signal, can subject surrounding air molecules and disassociate molecular bonds in oxygen and hydrogen molecules by subjecting the molecules through one or more electromagnetic fields or heating the molecules to the point where ionized molecules become increasingly electrically conductive. The PI generator head 125 can include a plurality of carbon tips. The carbon tips can include needle type or carbon-fiber brush type carbon tips. The PI generator head 125 can emit positive and negative ions through the carbon tips.

The power manager 130 can include a power management circuit that can receive power from the power supply 140 and convert an input voltage (e.g., 3.7 Volts) to one or more output voltages (e.g., 3.3 Volts and 12 Volts) to power the components in the PI generator circuit 100, including the controller 110 (e.g., 3.3 Volts), the PI driver 120 (e.g., 12 Volts), and the MD 170 (e.g., 12 Volts). The power manager 130 can include a DC/AC converter to convert direct current (DC) power to alternating current (AC) power. The power manager 130 can include an oscillator circuit.

The power supply 140 can include a battery or solar panel. The power supply 140 can include a rechargeable battery such as, for example, a lithium-ion (Li-ion) battery. The power supply 140 can include a DC power source. The power supply 140 can include a Li-ion battery package (e.g., 3.7 Volt), which can include a plurality (e.g., 4) Li-ion cells that can supply, for example, 10,000 mAH. The battery can include, for example, a 3.3V, 3.7V, 9V, or 12V battery, or any other voltage-level battery that is suitable to power the PI generator circuit 100.

Where a plurality of batteries are employed, the batteries can be included in a battery pack. For instance, a removable cartridge battery pack can be included that has a plurality (e.g., four) high capacity re-chargeable Li-ion cells, such as, for example, 18650 Li-Ion cells. The removable cartridge battery pack can include or be electrically connected to the I/O 160 or the CAPC 150 for charging safety.

The CAPC 150 can include an overcharge protection circuit such as, for example, a Zener-diode based circuit that can protect the power supply 140 from over charging, where the power supply 140.

The I/O interface 160 can include a power supply port. The I/O interface 160 can include a USB (Universal Serial Bus) port or socket, which can be configured to a DC power supply. The DC power supply can be applied, via the CAPC 150, to the power supply 140. A USB can be connected to the USB port in the I/O interface 160 for supplying power to external devices, such as mobile telephones.

The fan motor (FM) 175 can include a DC motor that is electrically connected to the MD 170. The MD 170 can include an integrated circuit (IC) on an IC board, which can be electrically connected to the power supply via the power manager 130. The FM 175 can be silent-running so that it does not make enough noise to be heard by nearby animals or occupants. The MD 170 can include a communication link connected to the controller 110 to receive a control signal to control an operating speed of the FM 175, or to stop or start supplying power to the FM 175.

As noted above, the display 185 can include an LCD, an LED array, a QLED array, or a plurality of LEDs (e.g., 4 LEDs), and, the user interface 185 can include a button, a keypad, a keyboard, a joy stick, a toggle switch, or VRS that can respond to voice commands. The display 180 can display the operating status of the PI generator 10, including an operating mode, status of the PI stream or power supply level. The user interface 185 can be actuated to turn ON/OFF the PI generator circuit 100, to control a PI mode of the PI generator circuit 100, or to check the status of the power supply such as a battery. The PI mode can include, for example, a distance mode, a fan speed mode, a PI stream rate mode, or a PI stream spread mode.

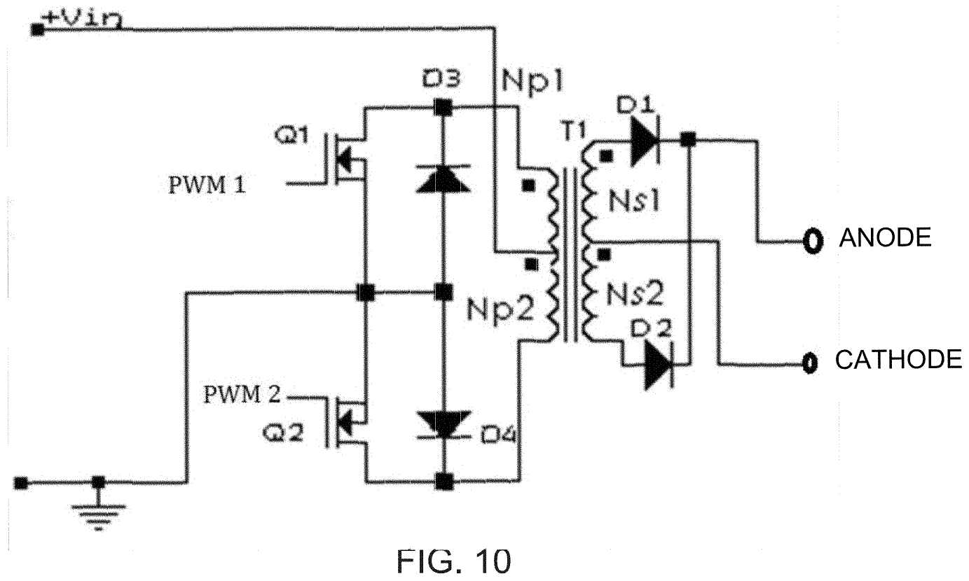

FIG. 10 shows an example of the high-voltage transformer that can be included in the PI driver 120 and connected to one or more canode pairings in the PI generator head 125 to provide electrical power to disassociate molecular bonds in air molecules. The transformer circuit can receive PWM 1 and PWM 2 signals from the controller 110 (shown in FIG. 9) at the gates of transistors Q1 and Q2, respectively, to control transformation of the input voltage Vin by the transformer T1 into output voltages ANODE and CATHODE, to be supplied to the PI generator head 125. The transformer circuit can include a plurality of diodes (e.g., Zener diodes) to control current direction.

As noted above the cathode-anode pairings in the PI generator head 125 can include carbon brushes or needles. For instance, in the embodiments of the PI generator 10 shown in FIGS. 5A, 5B and FIGS. 6-8, the PI generator head 125 can include one or more carbon brush canodes or one or more needle canodes. The needle type or brushes type carbon tip canodes, when supplied with the power signal from the PI driver 120, generate an electric field that is very strong where radius of conductive curvature is small such that more electric charges are concentrated on the sharp points. Other types of canodes can be included in the PI generator head 125, such as, for example, canodes having sharp or small-area tips that, when supplied with high voltage power, ionize molecules in the surrounding are.

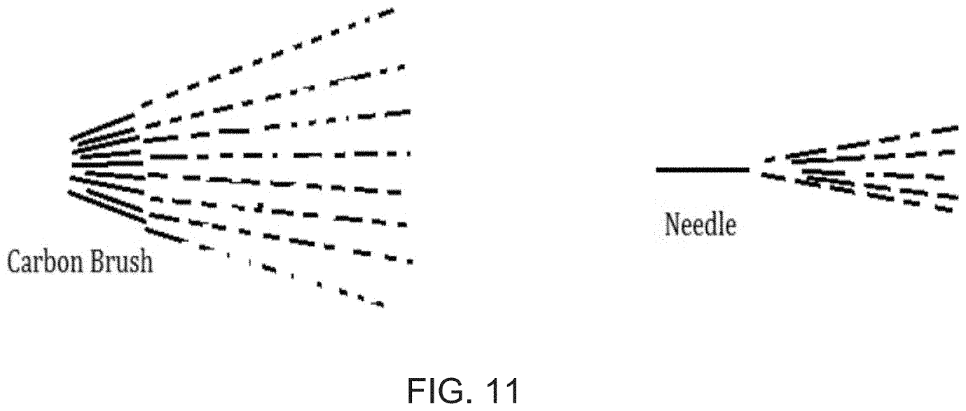

FIG. 11 illustrates an example of the difference in the amount of ion output between the brushes and needle types of canodes.

Since the brush type canodes can comprise numerous carbon fiber needles, the ions can be absorbed by plastic. Where the PI generator 10 housing comprises plastic, the ion stream ejection openings should be sufficiently large enough to provide unobstructed (or nearly unobstructed) passage of plasma ions into the surrounding area, in order to reduce ion absorption by the housing. The ion ejection openings should be formed to direct the PI stream (e.g., PI stream 40, shown in FIG. 1) for maximal coverage of the application domain 50. For instance, the ion ejection openings can be formed in a decline angle in order to blow plasma ions downward onto a hunter, where the PI generator 10 (e.g., embodiment shown in FIGS. 5A, 5B) is positioned above the hunter's head, as seen in FIG. 1, or formed at an angle that allows plasma ions to be blown upward into the area above the PI generator 10 where the PI generator 10 (e.g., embodiment shown in FIGS. 6-8) is placed on a horizontal planar surface, as seen in FIG. 2.

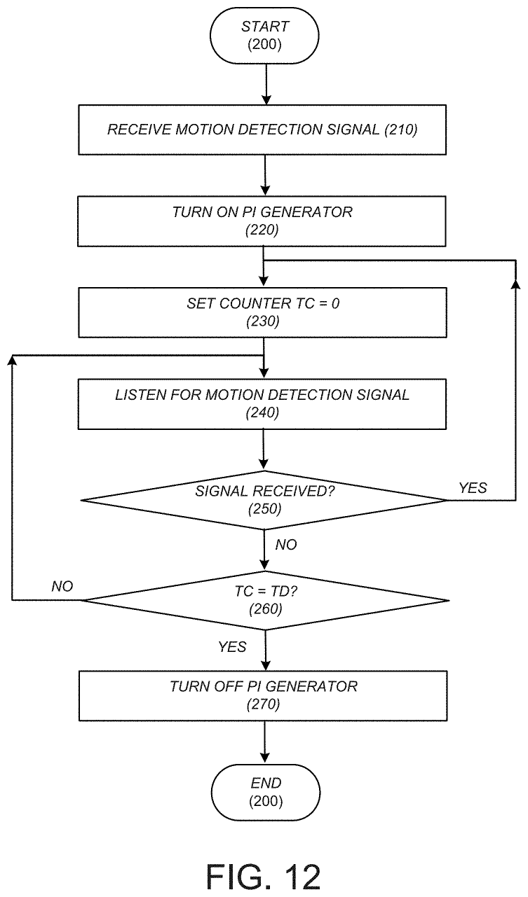

FIG. 12 shows an example of a de-scenting process 200 that can be carried out by the PI generator circuit 100 (shown in FIG. 9). The computer-readable medium in the PI generator 100 can include a computer program having sections of code or instructions that, when executed by the controller 110 (shown in FIG. 9), cause the steps in process 200 to be carried out by the PI generator circuit 100.

Referring to FIGS. 9 and 12 concurrently, the PI generator circuit 100 can turn ON (Step 220) after receiving a motion detection signal (Step 210) such as when an occupant walks into the room 2 (shown in FIG. 2). More specifically the controller 110 (shown in FIG. 9) can receive a motion detection signal from the motion detector 190 (shown in FIG. 9), which can sense, for example, IR energy and motion in the room 2.

Once turned ON, the controller 110 can instruct the PI driver 120 to power up the PI generator head 125 and begin ejecting a PI stream (e.g., PI stream 40 shown in FIG. 1). At this time the controller 110 can also set a timer counter TC to zero (TC=0) (Step 230). The timer counter can be set by the controller 110 (shown in FIG. 9), which can listen for additional motion detection signals from the motion detector 190 (Step 240). If a motion signal is received (YES at Step 250), then the controller 110 can reset the timer counter to zero (Step 220), otherwise (NO at Step 250) the controller 110 can determine whether the time counter has reached a preset time duration TD (Step 260). The time duration can be set by the controller 110 to, for example, 1 minute, 5 minutes, 10 minutes, or any other duration as will be understood by those skilled in the art.

Alternatively or additionally, the controller 110 can be configured to detect a motion magnitude vector for an object and, based on the motion magnitude vector, set or reset the time counter TC. The motion magnitude vector can include, for example, a direction of motion of the object, a speed or velocity of motion of the object, or a level of acceleration or deceleration of motion of the object in the application domain.

If the time counter is determined to have reached the preset time duration TD (YES at Step 260), then the controller 110 can instruct the PI driver 120 to stop powering the PI generator head 125 and turn OFF (Step 270). In a non-limiting embodiment, this can be done by the controller 110 terminating supply of the PWM signal to the PI driver 120.

The PI generator circuit 100 can remain in a standby mode until a motion detection signal is again received from the motion detector 190, at which point the process 200 can be repeated.

The terms “a,” “an,” and “the,” as used in this disclosure, means “one or more,” unless expressly specified otherwise.

The term “attachment mechanism,” as used in this disclosure, means an adhesive, a stitching, a button, a rivet, a hook-and-loop fastener, or any other device, composition, or mechanism practicable for the purposes intended herein, as understood by those skilled in the pertinent art.

The term “communicating device,” as used in this disclosure, means any device, hardware, firmware, or software that can transmit or receive an analog signal or a digital signal comprising data packets, instructions or data over a communication link. The communicating device can include a computing device. The communicating device can be portable or stationary.

The term “communication link,” as used in this disclosure, means a wired or wireless medium that conveys data or information between at least two points. The wired or wireless medium can include, for example, a metallic conductor link, a radio frequency (RF) communication link, an Infrared (IR) communication link, or an optical communication link. The RF communication link can include, for example, WiFi, WiMAX, IEEE 802.11, DECT, 0G, 1G, 2G, 3G, 4G or 5G cellular standards, or Bluetooth. A communication link can include, for example, an RS-232, RS-422, RS-485, or any other suitable interface.

The terms “computer” or “computing device,” as used in this disclosure, means any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a super computer, a personal computer, a laptop computer, a palmtop computer, a notebook computer, a desktop computer, a workstation computer, a server, a server farm, a computer cloud, or the like, or an array of processors, microprocessors, central processing units, general purpose computers, super computers, personal computers, laptop computers, palmtop computers, notebook computers, desktop computers, workstation computers, or servers.

The term “computer-readable medium,” as used in this disclosure, means any storage medium that participates in providing data (for example, instructions) that can be read by a computer. Such a medium can take many forms, including non-volatile media and volatile media. Non-volatile media can include, for example, optical or magnetic disks and other persistent memory. Volatile media can include dynamic random access memory (DRAM). Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium can include a “Cloud,” which includes a distribution of files across multiple (e.g., thousands of) memory caches on multiple (e.g., thousands of) computers.

Various forms of computer readable media can be involved in carrying sequences of instructions to a computer. For example, sequences of instruction (i) can be delivered from a RAM to a processor, (ii) can be carried over a wireless transmission medium, and/or (iii) can be formatted according to numerous formats, standards or protocols, including, for example, WiFi, WiMAX, IEEE 802.11, DECT, 0G, 1G, 2G, 3G, 4G, or 5G cellular standards, or Bluetooth.

The terms “including,” “comprising” and variations thereof, as used in this disclosure, mean “including, but not limited to,” unless expressly specified otherwise.

The term “network,” as used in this disclosure means, but is not limited to, for example, at least one of a personal area network (PAN), a local area network (LAN), a wireless local area network (WLAN), a campus area network (CAN), a metropolitan area network (MAN), a wide area network (WAN), a metropolitan area network (MAN), a wide area network (WAN), a global area network (GAN), a broadband area network (BAN), a cellular network, a storage-area network (SAN), a system-area network, a passive optical local area network (POLAN), an enterprise private network (EPN), a virtual private network (VPN), the Internet, or the like, or any combination of the foregoing, any of which can be configured to communicate data via a wireless and/or a wired communication medium. These networks can run a variety of protocols, including, but not limited to, for example, Ethernet, IP, IPX, TCP, UDP, SPX, IP, IRC, HTTP, FTP, Telnet, SMTP, DNS, ARP, ICMP.

Devices that are in communication with each other need not be in continuous communication with each other, unless expressly specified otherwise. In addition, devices that are in communication with each other may communicate directly or indirectly through one or more intermediaries.

Although process steps, method steps, algorithms, or the like, may be described in a sequential or a parallel order, such processes, methods and algorithms may be configured to work in alternate orders. In other words, any sequence or order of steps that may be described in a sequential order does not necessarily indicate a requirement that the steps be performed in that order; some steps may be performed simultaneously. Similarly, if a sequence or order of steps is described in a parallel (or simultaneous) order, such steps can be performed in a sequential order. The steps of the processes, methods or algorithms described herein may be performed in any order practical.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A de-scenting system that controls or eliminates a scent of an object in an application domain, the de-scenting system comprising:
a plasma ion generator that includes:
an air intake that receives air molecules;
a plasma ion generator head that disassociates molecular bonds in the received air molecules to create plasma ions;
a plasma ion ejector that directs the plasma ions from the plasma ion generator head in a predetermined direction; and
a fan that moves the surrounding air molecules to the air intake, and that moves the plasma ions through the plasma ion ejector in the predetermined direction,
wherein the plasma ions are applied to airborne particles in the application domain to eliminate or control the scent of the object.

2. The de-scenting system of claim 1, wherein the plasma ion generator further comprises a controller, a plasma ion driver, and a motion detector.

3. The de-scenting system of claim 2, wherein the controller instructs the plasma ion driver to supply power to the plasma ion generator head based on a motion detection signal received from the motion detector.

4. The de-scenting system of claim 1, further comprising a plasma ion generator circuit that includes:
a controller that generates a pulse width modulation (PWM) signal and a fan control signal;
a plasma ion driver that supplies power to the plasma ion generator head based on the pulse width modulation (PWM) signal; and
a motor driver that supplies power to the fan based on the fan control signal.

5. The de-scenting system of claim 1, wherein the plasma ions are applied to a surface of the object in the application domain, and wherein the plasma ions deodorize the object to camouflage the scent of the object.

6. The de-scenting system of claim 1, wherein the predetermined direction is downward when the plasma ion generator is positioned above the object.

7. The de-scenting system of claim 1, wherein the predetermined direction is upward when the plasma ion generator is located on a structural surface.

8. The de-scenting system of claim 1, wherein the plasma ion generator comprises an attachment member that attaches to a tree.

9. The de-scenting system of claim 1, wherein the plasma ion generator head includes a canode pairing.

10. The de-scenting system of claim 9, wherein the canode pairing comprises a needle type or brush type anode or cathode.

11. The de-scenting system of claim 9, wherein the canode pairing comprises carbon fiber.

12. The de-scenting system of claim 1, wherein the object is a person, clothing or equipment employed in a hunting sport.

13. A de-scenting system that controls or eliminates a scent in an application domain, the de-scenting system comprising:
a plasma ion generator that includes:
a motion detector that detects movement of an object in or near the application domain and generates a motion detection signal;
an air intake that receives air molecules;
a plasma ion generator head that disassociates molecular bonds in the received air molecules to create plasma ions;
a plasma ion ejector that directs the plasma ions from the plasma ion generator head in a predetermined direction;
a plasma ion driver that supplies power to the plasma ion generator head;
a controller that generates a pulse width modulation (PWM) signal to drive the plasma ion driver based on the motion detection signal; and
a fan that moves the surrounding air molecules to the air intake, and that moves the plasma ions through the plasma ion ejector in the predetermined direction,
wherein the plasma ions are applied to airborne particles in the application domain to eliminate or control the scent.

14. The de-scenting system of claim 13, wherein the predetermined direction is upward when the plasma ion generator is located on a structural surface.

15. The de-scenting system of claim 13, wherein the plasma ion generator head includes a canode pairing.

16. The de-scenting system of claim 15, wherein the canode pairing comprises a needle type or brush type anode or cathode.

17. The de-scenting system of claim 15, wherein the canode pairing comprises carbon fiber.

18. A method for controlling or eliminating scent an object in an application domain, the method comprising:
receiving air molecules through an air intake;
disassociating molecular bonds in the received air molecules to create plasma ions;
directing the plasma ions in a predetermined direction;
moving the plasma ions through a plasma ion ejector in the predetermined direction; and
applying the plasma ions to airborne particles in the application domain to eliminate or control the scent of the object.

19. The method of claim 18, further comprising applying the plasma ions to airborne particles in the application domain to de-scent the object to camouflage the scent of the object from wild game.

20. The method of claim 18, wherein the predetermined direction is downward when the plasma ion generator is positioned above the object.

21. The method of claim 18, further comprising detecting motion of the object in the application domain and applying the plasma ions to airborne particles in the application domain when motion of the object is detected.
